(19) United States
(12) Patent Application Publication
(10) Pub. No.: US 2003/0233141 A1
(43) Pub. Date: Dec. 18, 2003

(54) Stent coated with stent graft and method therefor

(76) Inventor: Henry M. Israel, Bnei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/167,695

(22) Filed: Jun. 13, 2002

(51) Int. Cl. A61F 2/06

(52) U.S. Cl. 623/1.15; 623/1.46

(57) ABSTRACT

A method for manufacturing a stent comprising coating a stent body in an elastomeric material to form an elastomeric film coating over exterior and interior surfaces of the stent body over a longitudinal length thereof, the stent body comprising a plurality of structural members connected to one another with openings formed between at least a portion of the structural members, and the elastomeric film coating extending over the openings and the structural members. The coating may be cured afterwards. A stent constructed with this method is also described.

### FIELD OF THE INVENTION

The present invention relates generally to stents and stent grafts, and particularly to a stent coated with a stent graft.

### BACKGROUND OF THE INVENTION

Stents, such as vascular stents, may be deployed at a narrowed site in a vessel of a patient for widening the vessel lumen and circumferentially supporting the vessel wall. The stent may typically have a small cross-sectional diameter and/or profile for introducing the stent into the affected vessel lumen, and the stent may be expandable after placement in the vessel lumen.

There are many kinds of constructions of stents, such as but not limited to, bent-wire stents, wire mesh stents, flat metal sheets formed into a stent shape, metal cannula stents with flexible regions or articulations, or expandable stents (e.g., made of a shape memory alloy, such as nickel-titanium alloy).

Portions of stents have been coated with different materials. For example, the metal wires of some stents have been coated with polytetrafluoroethylene (PTFE) to reduce friction during insertion of the stent in a lumen or to enhance or modify flows characteristics of a fluid (e.g., blood) flowing through the stent. As another example, the metal wires of some stents have been coated with radiopaque material to enhance the visibility of the stent in fluoroscopic or X-ray imaging. Coatings may protect against scratches, flaking and galvanic corrosion, for example.

Stent grafts are materials used to cover the stent. Grafts may be used for various purposes, such as but not limited to, enhancement of flow properties of fluids such as blood flowing through the stent, or protection of tissues against injury upon introduction of the stent in a body lumen. Stent grafts may be made in a variety of manners. For example, stent grafts are known that comprise a plastic flexible sleeve slipped over the stent and bonded or sewn thereto.

### SUMMARY OF THE INVENTION

The present invention seeks to provide an improved stent coated with a stent graft. In one example, the stent may be manufactured by coating the stent in an elastomeric (for example, an elastomeric polymer, such as silicone rubber) dispersion, wherein the elastomeric material cures and forms interior and exterior coatings on the stent. Apertures in the stent graft may be formed during or after the coating process.

There is thus provided in accordance with a an embodiment of the invention a method for manufacturing a stent comprising coating a stent body in an elastomeric material to form an elastomeric film coating over exterior and interior surfaces of the stent body over a longitudinal length thereof, the stent body comprising a plurality of structural members connected to one another with openings formed between at least a portion of the structural members, and the elastomeric film coating extending over the openings and the structural members. The coating may be cured afterwards.

In accordance with an embodiment of the invention the elastomeric material comprises an elastomer dispersed in a solvent, such as but not limited to, a silicone elastomer dispersed in xylene.

Further in accordance with a an embodiment of the invention a mandrel is placed in an interior portion of the stent body prior to coating the stent body in the elastomeric material.

Still further in accordance with an embodiment of the invention the elastomeric material covers the interior surface of the stent body between the stent body and the mandrel.

In accordance with a an embodiment of the invention a smoother elastomeric film coating is formed over the interior surface of the stent body than over the exterior surface thereof.

Further in accordance with an embodiment of the invention one or more apertures are formed through a thickness of the elastomeric film coating.

There is also provided in accordance with a an embodiment of the invention a stent comprising a stent body comprising a plurality of structural members connected to one another with openings formed between at least a portion of the structural members, and a stent graft comprising an elastomeric film coating coated over exterior and interior surfaces of the stent body over a longitudinal length thereof, the elastomeric film coating extending over the openings and the structural members. The elastomeric material may include a radiopaque substance or a medicinal substance.

### BRIEF DESCRIPTION OF THE DRAWINGS

The present invention will be understood and appreciated more fully from the following detailed description taken in conjunction with the drawings in which:

FIG. 1 is a simplified pictorial illustration of a stent prior to coating with an elastomeric material, in accordance with an embodiment of the invention;

FIG. 2 is a simplified pictorial illustration of the stent of FIG. 1 during coating with the elastomeric material, in accordance with an embodiment of the invention; and

FIGS. 3 and 4 are simplified pictorial and sectional illustrations, respectively, of the stent of FIG. 1 coated with the elastomeric material, in accordance with an embodiment of the invention, FIG. 4 being taken along lines IV-IV in FIG. 3.

### DETAILED DESCRIPTION OF A PREFERRED EMBODIMENT

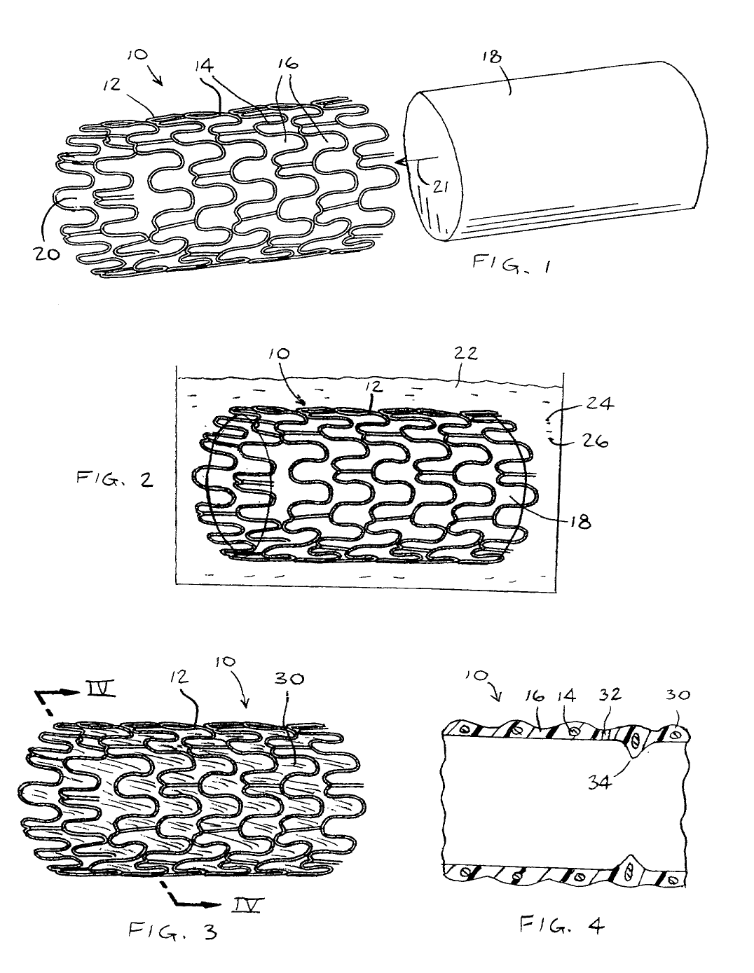

Reference is now made to FIG. 1, which illustrates a stent 10, comprising a stent body 12 made of any suitable biocompatible, and preferably flexible material, such as but not limited to, a shape memory alloy (e.g., a nickel-titanium alloy) or a stainless steel alloy. Stent body 12 may be constructed of a plurality of structural members 14 connected to one another with openings 16 formed between at least some of structural members 14. Structural member 14 may comprise, without limitation, a strut, radial element, or longitudinal element, and may be made from wire, cut stock, or other materials. The invention is not limited to any particular stent construction. A mandrel 18 may be placed in an interior portion 20 of stent body 12, as indicated by arrow 21 in FIG. 1.

Reference is now made to FIG. 2. After mandrel 18 has been inserted in stent body 12, stent body 12 may be coated with an elastomeric material 22. Stent body 12 may be dipped in elastomeric material 22, or elastomeric material 22 may be otherwise applied on stent body 12. Elastomeric material 22 may cover the interior surface of stent body 12 between stent body 12 and mandrel 18. Elastomeric material 22 may comprise an elastomer dispersed in a solvent. For example, elastomeric material 22 may comprise a two-component silicone elastomer dispersed in xylene, such as the MED-66xx silicone dispersion elastomers (e.g., those approved for unrestricted use for over 29 days) commercially available from NuSil Technology, Carpinteria, Calif., USA. Other dispersion silicone rubbers or polymers may also be used. After coating, elastomeric material 22 may be cured, such as in accordance with manufacturer's instructions.

Coating stent body 12 with elastomeric material 22 may form a stent graft comprising an elastomeric film coating 30 over exterior and interior surfaces of stent body 12 over a longitudinal length thereof, as seen in FIG. 3. The elastomeric film coating 30 may extend over openings 16 and structural members 14. The engineering properties of elastomeric material 22 may be selected to control viscosity, coating ability, surface tension and other factors that may affect how elastomeric material 22 covers the openings 16 (e.g., what size opening may be covered) and structural members 14 (e.g., what thickness of coating is obtained). The stent graft so obtained may be flexible, contractible, expandable and stretchable. For example, the stent with the stent graft of the invention may be compressed for introduction into a body lumen with a very small diameter catheter introducer and expanded after placement in the lumen to a much larger diameter.

As seen in FIG. 4, the elastomeric film coating 30 formed over the interior surface of stent body 12 may be smooth, if mandrel 18 is smooth. The elastomeric film coating 30 formed over the exterior surface of stent body 12 may be as smooth, smoother or less smooth. In the illustrated embodiment, the elastomeric film coating 30 formed over the exterior surface of stent body 12 is wavy, conforming to the contour of the outer surface of the stent 10, and not as smooth as the interior coating.

In accordance with an embodiment of the invention, elastomeric film coating 30 may be porous or non-porous.

In accordance with an embodiment of the invention, one or more apertures 32 may be formed through a thickness of elastomeric film coating 30, as seen in FIG. 4. Apertures 32 may be formed by pricking the coating 30 after curing thereof. Alternatively, apertures 32 may be formed during the casting or coating process, by providing mandrel 18 with small spike-like protrusions that protrude through stent body 12. The elastomeric material 22 may be wiped over the exterior surface of the stent body 12, with the result that no elastomeric material remains at the extremities of the protrusions. Such a technique may form apertures 32 in the cured elastomeric film coating 30 at the sites of the protrusions. It is appreciated that other techniques may be used to form apertures 32.

Elastomeric material 22 may comprise different additives for enhancing physical, mechanical, medicinal or other properties thereof. For example, elastomeric material 22 may be mixed with a radiopaque substance 24 (FIG. 2) for enhancing the visibility of stent 10 in fluoroscopic or X-ray imaging. As another example, elastomeric material 22 may be mixed with a medicinal substance 26, for introduction into a patient, such as by means of dissolving, absorption, osmosis or iontophoresis, for example. Alternatively, these substances may be applied over the elastomeric material 22 after curing thereof.

Stent body 12 does not have to be straight, and may be bent or otherwise formed to a variety of shapes and cross-sections. For example, as seen in FIG. 4, stent body 12 may be formed with a non-uniform cross-section 34, such as but not limited to, a generally cylindrical shape with a nozzle, venturi or constriction, which may serve to control flow of a substance in the lumen where stent 10 is positioned, such as reducing turbulent flow. The elastomeric film coating 30 may conform to the shape of the non-uniform cross-section 34. Alternatively, the elastomeric film coating 30 may be used to form the non-uniform cross-section 34 to control flow of the substance in the lumen.

The elastomeric film coating 30 may be uniform and homogeneous. Alternatively, the elastomeric film coating 30 may comprise a non-homogeneous coating comprising different elastomeric materials with different properties, such as but not limited to, different stretching properties, flexibility, thermal conductivity, tensile strength and viscosity, to name a few. Different portions of the stent may be coated with different kinds of elastomeric materials.

It will be appreciated by person skilled in the art, that the present invention is not limited by what has been particularly shown and described herein above. Rather the scope of the present invention is defined only by the claims that follow:

What is claimed is:

1. A method for manufacturing a stent comprising:
coating a stent body with an elastomeric material to form an elastomeric film coating over exterior and interior surfaces of said stent body over a longitudinal length thereof, said stent body comprising a plurality of structural members connected to one another with openings formed between at least a portion of said structural members, and said elastomeric film coating extending over said openings and said structural members.

2. The method according to claim 1, further comprising curing said elastomeric film coating.

3. The method according to claim 1, wherein said elastomeric material comprises an elastomer dispersed in a solvent.

4. The method according to claim 3, wherein said elastomeric material comprises a silicone elastomer dispersed in xylene.

5. The method according to claim 1, wherein a mandrel is placed in an interior portion of said stent body prior to coating said stent body with said elastomeric material.

6. The method according to claim 5, wherein said elastomeric material covers the interior surface of said stent body between said stent body and said mandrel.

7. The method according to claim 1, further comprising forming a smoother elastomeric film coating over the interior surface of said stent body than over the exterior surface thereof.

8. The method according to claim 1, further comprising forming at least one aperture through a thickness of said elastomeric film coating.

9. A stent comprising:
a stent body comprising a plurality of structural members connected to one another with openings formed between at least a portion of said structural members; and
a stent graft comprising an elastomeric film coating coated over exterior and interior surfaces of said stent body over a longitudinal length thereof, said elastomeric film coating extending over said openings and said structural members.

10. The stent according to claim 9, wherein said elastomeric material comprises an elastomer dispersed in a solvent.

11. The stent according to claim 10, wherein said elastomeric material comprises a silicone elastomer dispersed in xylene.

12. The stent according to claim 9, wherein the elastomeric film coating is smoother over the interior surface of said stent body than over the exterior surface thereof.

13. The stent according to claim 9, wherein at least one aperture is formed through a thickness of said elastomeric film coating.

14. The stent according to claim 9, wherein said elastomeric material includes a radiopaque substance.

15. The stent according to claim 9, wherein said elastomeric material includes a medicinal substance.

16. The stent according to claim 9, wherein said stent body comprises a non-uniform cross-section.

17. The stent according to claim 9, wherein said elastomeric film coating comprises a non-uniform cross-section.

18. The stent according to claim 9, wherein said elastomeric film coating is homogeneous.

19. The stent according to claim 9, wherein said elastomeric film coating is non-homogeneous.

20. The stent according to claim 9, wherein said elastomeric film coating comprises different elastomeric materials.

21. The stent according to claim 9, wherein said elastomeric film coating is porous.

22. The stent according to claim 9, wherein said elastomeric film coating is nonporous.
